(19) United States
(12) Patent Application Publication
(10) Pub. No.: US 2019/0048389 A1
(43) Pub. Date: Feb. 14, 2019

(54) CULTURE MEDIUM WITH TWO LAYERS FOR DETECTING MICROORGANISMS

(71) Applicant: JNC CORPORATION, TOKYO (JP)

(72) Inventors: Hajime TERAMURA, Kanagawa (JP); Mihoko IWASAKI, Kanagawa (JP); Kojiro SOTA, Kanagawa (JP)

(73) Assignee: JNC CORPORATION, TOKYO (JP)

(21) Appl. No.: 16/076,694

(22) PCT Filed: Oct. 3, 2016
(86) PCT No.: PCT/JP2016/004455

§ 371 Date: Aug. 8, 2018

(30) Foreign Application Priority Data
Feb. 9, 2016 (JP) 2016-022767

(51) Int. Cl. C12Q 1/04

(52) U.S. Cl. CPC C12Q 1/045

(57) ABSTRACT

To provide a method in which staining of a culture medium itself can be suppressed even if an enzyme that can decompose an enzyme substrate to be decomposed by an enzyme of microorganisms inherently exists in an analyte, and target microorangisms can be detected as stained colony with high accuracy, and an enzyme substrate culture medium therefor. A culture medium for detecting microorganisms includes a first layer in which a gelling agent and an enzyme substrate are contained, and a second layer in which a gelling agent is contained and the enzyme substrate is not contained to be laminated adjacently to the first layer, wherein the enzyme substrate is a compound from which a dye compound can be released.

### TECHNICAL FIELD

The present invention relates to a culture medium for detecting microorganisms as colored or fluorescent colony.

### BACKGROUND ART

When specific microorganisms are detected, a selective medium fitted to target microorganisms, such as a culture medium containing a selective agent such as an antibiotic and a surfactant, a culture medium adjusted to specific pH, and a culture medium adjusted to a specific glucose concentration, is generally used.

Moreover, a method (enzyme substrate method) is also utilized, in which a substrate compound to be decomposed by an enzyme specifically possessed by the target microorganisms is incorporated into a culture medium, and the target microorganisms grown on the culture medium are detected as colony stained by a dye compound released from the substrate in receiving decomposition by the enzyme. The culture medium to be used in such a method is called “enzyme substrate culture medium,” and development has been made on various substrate compounds with which a colored or fluorescent dye compound is bonded, and proposals have been made on the enzyme substrate culture media for wide range of strains in food and drink or clinical fields (Patent literature No. 1 and the like).

An enzyme substrate incorporated into the enzyme substrate culture medium is decomposed by an enzyme possessed in the target microorganisms. However, the enzyme that can decompose the enzyme substrate in the enzyme substrate culture medium is contained even in an analyte of fermented food such as cheese and an analyte containing a large amount of tissues such as raw meat in several cases. When such an analyte is provided as a test sample, the enzyme substrate is decomposed by the enzyme inherently existing in the analyte, and the culture medium is wholly stained by the dye compound, regardless of existence or nonexistence of the target microorganisms. Therefore, a problem occurs in which distinguishing of the colony of the target microorganisms becomes difficult.

For example, Coliform bacilli are known to specifically possess β-galactosidase, and when Coliform bacilli are detected by the enzyme substrate method, such a culture medium is used as the culture medium containing the enzyme substrate from which the dye compound such as 5-bromo-4-chloro-3-indoxlyl-β-D-galactopyranoside (X-GAL) can be released by decomposition by β-galactosidase. However, when an attempted is made on detecting Coliform bacilli from a fermented food analyte such as cheese by using the enzyme substrate culture medium, a large amount of β-galactosidase inherently existing in the analyte and derived from Lactobacilli exists, and therefore the enzyme substrate in the culture medium for which the analyte is provided as a sample is decomposed, and the culture medium is wholly stained by the dye compound. Therefore, distinguishing of stained colony of Coliform bacilli becomes difficult.

In recent years, development has been made on various sheet-shaped simple culture media or the like in order to achieve a rapid culturing test, in which the culture medium is in a dry state during storage, but a gelling agent is swollen with moisture contained in the analyte upon applying the analyte as a sample (Patent literature Nos. 2 to 5 and the like). In a case where such a simple culture medium is used, the enzyme inherently existing in the analyte easily penetrates into the culture medium. Therefore, a problem in the enzyme substrate method becomes further significant in comparison with a case when an ordinal agar medium is used.

### CITATION LIST

### Patent Literature

Patent literature No. 1: JP 2002-537852 A.

Patent literature No. 2: WO 97/24432 A.

Patent literature No. 3: JP H10-501129 A.

Patent literature No. 4: JP H9-19282 A.

Patent literature No. 5: JP 2015-204845 A.

### SUMMARY OF INVENTION

### Technical Problem

In view of such a situation, the invention is contemplated for providing a method in which, even if an enzyme that can decompose an enzyme substrate to be decomposed by an enzyme of microorganisms inherently exists in an analyte, staining of a culture medium itself can be suppressed, and as a result, target microorganisms can be detected as stained colony with high accuracy, and an enzyme substrate culture medium therefor.

### Solution to Problem

The present inventors have diligently continued to conduct study in order to solve the problems as described above. As a result, the present inventors have found that an enzyme inherently existing in an analyte to be provided as a sample can be prevented from being brought into contact with an enzyme substrate by laminating a culture medium containing no enzyme substrate onto an enzyme substrate culture medium, and as a result, staining of the culture medium itself can be suppressed, and have completed the invention.

More specifically, the invention includes the items described below.

Item 1.

A culture medium for detecting microorganisms, including:

a first layer in which a gelling agent and an enzyme substrate are contained, and

a second layer in which a gelling agent is contained and the enzyme substrate is not contained to be laminated adjacently to the first layer,

wherein the enzyme substrate is a compound from which a dye compound can be released.

Here, as the invention of item 1, the following aspects are preferred.

Item 1-1.

The culture medium according to item 1, wherein the second layer inhibits or retards bringing an enzyme in the analyte into physical contact with the enzyme substrate existing in the first layer.

Item 1-2.

The culture medium according to item 1, wherein the enzyme substrate is an enzyme substrate from which an indoxlyl-based dye compound such as
5-bromo-4-chloro-3-indoxlyl-β-D-galactopyranoside can be released, or an enzyme substrate from which a fluorescent compound such as
4-methylumbelliferyl-β-D-galactopyranoside can be released.

Item 2.

The culture medium according to item 1, wherein the gelling agent contains one kind or two or more kinds selected from the group of agar, guar gum, xanthan gum, locust bean gum, gellan gum, polyvinyl alcohol, alkylcellulose, carboxyalkylcellulose and hydroxyalkylcellulose.

Item 3.

A material for detecting microorganisms, including:

the culture medium according to item 1 or 2, and

a third layer in which a porous material is contained to be laminated adjacently to the second layer on a side opposite to a side to which the first layer is adjacent.

Item 4.

A method for detecting microorganisms, including a step of inoculating an analyte into the second layer in the culture medium according to item 1 or 2, a step of culturing the microorganisms contained in the analyte, and a step of detecting stained colony of the microorganisms,

wherein the enzyme substrate is a substrate to be decomposed by an enzyme of the microorganisms.

Item 5.

A method for suppressing staining of a culture medium, including a step of inoculating an analyte into the second layer in the culture medium according to item 1 or 2, and a step of culturing microorganisms contained in the analyte, wherein the enzyme substrate is a substrate to be decomposed by an enzyme of the microorganisms and an enzyme contained in the analyte.

### Advantageous Effects of Invention

If a culture medium according to the invention is used, even if an enzyme that can decompose an enzyme substrate to be decomposed by an enzyme of microorganism inherently exists in an analyte, staining of the culture medium itself can be suppressed, and as a result, target microorganisms can be detected as stained colony with high accuracy. In particular, the culture medium according to the invention is useful when the medium is prepared into a form of a simple culture medium being in a dry state before use to realize rapid and simple detection of the target microorganisms.

### BRIEF DESCRIPTION OF DRAWINGS

FIG. 1 is photographs showing colony (K. oxytoca) on culture media in Comparative Example 1 and Example 1.

FIG. 2 is photographs showing colony (E. coli) on culture media in Comparative Example 1 and Example 1.

### DESCRIPTION OF EMBODIMENTS

A culture medium according to the invention has features of having a first layer and a second layer.

Moreover, in the first layer, a gelling agent and an enzyme substrate are contained. On the other hand, in the second layer, a gelling agent is contained and the enzyme substrate is not contained.

The second layer is laminated adjacently to the first layer. Here, “laminated” means that the second layer at least partially covers the first layer, which is sufficient, but the second layer wholly covers the first layer, which is preferred. The first layer and the second layer are laminated adjacently to each other. Thus, when the analyte is provided as a sample from a side of the second layer as in the method according to the invention as mentioned later, the second layer prevents (inhibits or retards) bringing of the enzyme in the analyte into contact with the enzyme substrate contained in the first layer, and staining of the culture medium itself can be suppressed. Therefore, as a result, the target microorganisms can be clearly distinguished as stained colony. In addition, “enzyme in the analyte” and “enzyme contained in the analyte” herein means the enzyme inherently possessed by the analyte, which is distinguished from the enzyme of the target microorganisms.

The gelling agent in the invention means a substance that causes swelling and gelling by moisture, and plays a role of a matrix for shaping the culture medium. More specifically, the culture medium according to the invention is ordinarily solid (including a gel-form).

The gelling agent is ordinarily a polymer compound and may be a substance to be generally used for a solid medium for culturing the microorganisms, such as a viscosity-improving polysaccharide and an absorbent polymer. Specific examples thereof include agar, guar gum, xanthan gum, locust bean gum, gellan gum, polyvinyl alcohol, alkylcellulose such as methylcellulose and ethylcellulose, carboxyalkylcellulose such as carboxymethylcellulose and carboxyethylcellulose, and hydroxyalkylcellulose such as hydroxymethylcellulose and hydroxyethylcellulose, and a mixture in combination of one kind or two or more kinds therefrom can be used. Moreover, with regard to magnitude (average molecular weight, degree of polymerization or the like) of the polymer compound, the compound in the general range when the compound is used in the solid culture medium for culturing the microorganisms. For example, polyvinyl alcohol having a weight average molecular weight of preferably 5,000 to 200,000 and a degree of saponification of preferably 75 to 99%, and further preferably 85 to 90% can be used.

Moreover, a kind of the gelling agent to be incorporated into the first layer and the second layer may be identical or different. However, the gelling agent of the same kind is preferred from a viewpoint of compatibility (affinity) between two layers.

A concentration of the gelling agent in the first layer and the second layer during use (during growth of the microorganisms, the same shall apply hereinafter) may be adjusted to a general range when the gelling agent is used in the solid culture medium for culturing the microorganisms. For example, when polyvinyl alcohol having a weight average molecular weight of 5,000 to 200,000 and a degree of saponification of 75 to 99% is used therefor, the concentration during use is preferably 140 to 300 g/L, and further preferably 160 to 260 g/L. Moreover, for example, when agar having a weight average molecular weight of 10,000 to 1,000,000 is used, the concentration during used is preferably 5 to 30 g/L, and further preferably 10 to 20 g/L. The culture medium can be easily handled and shaped by adjusting the content to such a level. The concentrations of the gelling agent in the first layer and the second layer during use may be identical or different. However, the concentrations are preferably in an identical or approximate range from a viewpoint of compatibility (affinity) between the two layers.

An amount of the gelling agent in the second layer is not particularly limited, as long as such an amount is applied in which the enzyme in the analyte to be provided as the sample to the culture medium is prevented from being brought into contact with the enzyme substrate contained in the first layer. For example, when polyvinyl alcohol having a weight average molecular weight of 5,000 to 200,000 and a degree of saponification of 75 to 99% is used, the amount of the gelling agent in the second layer is preferably 0.5 to 5 g/m², and further preferably 0.5 to 2 g/m². Moreover, for example, when agar having a weight average molecular weight of 10,000 to 1,000,000 is used, the amount of the gelling agent in the second layer is preferably 15 to 75 g/m², and further preferably 15 to 30 g/m².

Moreover, a thickness of the second layer during use is not particularly limited, as long as such a thickness is applied at which the enzyme in the analyte to be provided as the sample to the culture medium can be prevented from being brought into contact with the enzyme substrate contained in the first layer. For example, the thickness of a sheet-shaped dry simple culture medium as mentioned later is preferably 0.001 to 0.1 mm, and further preferably 0.01 to 0.1 mm during use, and the thickness in the form of the agar medium is preferably 0.1 to 5 mm, and further preferably 0.5 to 2 mm.

The enzyme substrate in the invention is a compound from which a dye compound can be released in receiving decomposition of the substrate.

Here, the dye compound may be any of a colored compound under visible light and a compound emitting color fluorescence. Specific examples of a functional group in the compound that can be released as the colored compound under visible light include a 5-bromo-4-chloro-3-indoxyl group, and released 5-bromo-4-chloro-3-indole is subjected to oxidation condensation into 5,5′-dibromo-4,4′-dichloro-indigo to show blue color. Specific examples of the functional group in the compound that can be released as the compound emitting color fluoresce include a 4-methylumbelliferyl group, and released 4-methylumbelliferone emits fluorescence under irradiation with ultraviolet light.

Moreover, the enzyme substrate in the invention is ordinarily a substrate to be decomposed by the enzyme of the target microorganisms the detection of which is desired by using the culture medium. In the enzyme substrate, the dye compound is ordinarily bonded with a recognition site through a cleavage site by the enzyme. The enzyme substrate contained in the first layer is decomposed by the enzyme. Thus, the dye compound is released, and grown target microorganisms are detected as colored colony or colony stained by fluorescence. Therefore, such colony can be easily distinguished on the culture medium.

Moreover, the enzyme substrate in the invention may be a substrate to be decomposed by the enzyme contained in the analyte to be provided as the sample to the culture medium. As mentioned above, in the culture medium according to the invention, the enzyme substrate is contained in the first layer, and no enzyme substrate is contained in the second layer, and the second layer inhibits the enzyme in the analyte from being brought into contact with the enzyme substrate contained in the first layer. Therefore, even when the enzyme that can decompose the enzyme substrate is contained in the analyte, the culture medium itself is not stained, and the stained colony of the target microorganisms can be clearly distinguished.

Specific examples of the enzyme substrates include
5-bromo-4-chloro-3-indoxlyl-β-D-galactopyranoside (X-GAL) and
5-bromo-4-chloro-3-indoxyl-β-D-glucuronic acid in the case where the target microorganisms are Coliform bacilli, 5-bromo-4-chloro-3-indoxyl-phosphoric acid (X-phos) in the case of Staphylococcus aurei,
5-bromo-4-chloro-3-indoxlyl-β-D-glucopyranoside (X-GLUC) in the case of Enterococcus, and 5-bromo-4-chloro-3-indoxyl-acetic acid and
5-bromo-4-chloro-3-indoxyl-butyric acid in the case of Fungi, and all of which can be preferably used, respectively.
   A concentration of such enzyme substrates in the first layer during use only needs to be adjusted to a general range in which the enzyme substrate is used in the solid culture medium for detecting the microorganisms. For example, the concentration thereof during use is preferably 0.01 to 1.0 g/L, and further preferably 0.2 to 0.6 g/L.

In the first layer and the second layer each in the culture medium according to the invention, a selective substance, an antibacterial substance, a nutritional ingredient, inorganic salts, a saccharide, a viscosity improver, a pH adjuster or the like may be arbitrarily contained, in addition to the above-described ingredients. Moreover, compositions may be identical or different in the first layer and the second layer, excluding existence or nonexistence of a color-developing enzyme substrate.

Specific examples of the selective substance include an antibiotics and a surfactant such as sodium dodecyl sulfate (SDS), Tween 80 and bile salt such as sodium cholate. In particular, incorporation of the surfactant into the second layer is reasonable from viewpoints of modifying the enzyme contained in the analyte or suppressing growth of Gram-positive bacteria.

Specific examples of the antibacterial substance include polylysine, protamine sulfate, glycine and sorbic acid.

As the nutritional ingredient, peptone, an animal meat extract, a yeast extract or a fish meat extract is preferred, for example.

Specific examples of the inorganic salts include inorganic acid metal salt such as sodium chloride and sodium thiosulfate, and organic acid metal salt such as sodium pyruvate, ferric ammonium citrate and sodium citrate.

Specific examples of the saccharide include glucose, lactose, sucrose, xylose, cellobiose and maltose.

Specific examples of the viscosity improver include starch and a derivative thereof, hyaluronic acid, an acrylic acid derivative, polyether and collagen.

Specific examples of the pH adjuster include sodium carbonate and sodium hydrogencarbonate.

In the culture medium according to the invention, from a viewpoint of growth of the target microorganisms, pH during use is preferably 6.0 to 8.0, and further preferably 6.5 to 7.5.

A form of the culture medium according to the invention is not particularly limited, and the culture medium can be prepared into a sheet-shaped simple dry culture medium or the like, in addition to a form in which the culture medium is cast into a petri dish or the like and solidified therein.

Specific example of the sheet-shaped dry simple culture medium include a sheet-shaped culture medium having a configuration formed by laminating a layer in which a porous material is contained and a layer in which a gelling agent is contained and including the layers, as described in WO 97/24432 A. In the above case, a material for detecting the microorganisms only needs to be formed by applying the layer in which the gelling agent is contained as the culture medium according to the invention, and the layer in which the porous material is contained as the third layer adjacent to the second layer on the side opposite to the side to which the first layer is adjacent in the culture medium according to the invention, and including the above layers. More specifically, the material for detecting the microorganisms as related to the invention includes a configuration in which the first layer, the second layer and the third layer are adjacently laminated in the above order. When the material is used in the method according to the invention as mentioned later, the analyte may be added from the side of the third layer, and then provided as the sample to the side of the second layer.

Specific examples of the porous material contained in the third layer include a knitted or woven fabric, a nonwoven fabric, a porous film and sponge each formed of synthetic fibers, semisynthetic fibers, natural fibers and inorganic fibers. Porous ceramics may also be used. Specific examples of the synthetic fibers include fibers of nylon, polyacrylonitrile, polyvinyl alcohol, an ethylene-vinyl acetate copolymer, polyester that may be subjected to hydrophilic treatment, polyolefin that may be subjected to hydrophilic treatment and polyurethane. Specific examples of the semisynthetic fibers include fibers of rayon. As the natural fibers, fibers of wool, silk, cotton, cellulose, pulp or the like are preferred.

In particular, a kitted or woven fabric, a nonwoven fabric or the like in which adjustment of unit weight or air permeability is easy is preferred, and a nylon meltblown nonwoven fabric prepared by a meltblown manufacturing method according to which fine fibers can be comparatively easily obtained, or an ultrafine fiber nonwoven fabric manufactured from splittable fibers is further preferred.

Moreover, a unit weight of the porous material is preferably 50 to 90 g/m², and further preferably 55 to 80 g/m². If the unit weight is within the above range, moisture retention capability in a microorganism culturing material can be sufficiently easily secured, no liquid sample (analyte) overflows from the third layer, and the third layer and the second layer can be sufficiently integrated.

Moreover, air permeability of the porous material is preferably 7 to 24 cm/sec (70 to 240 L/(m² ·sec)), further preferably 8 to 20 cm/sec, and still further preferably 10 to 18 cm/sec. If the air permeability is within the above range, moisture is uniformly easily distributed into the second layer and the first layer upon adding the liquid sample (analyte) thereto, and the microorganisms are uniformly easily cultured. Moreover, fixing properties when the gelling agent contained in the second layer and the first layer is dissolved and swollen are sufficiently easily secured. In addition, the air permeability is measured by Frazier Type Method specified in JIS L1096 8.26.

The culture medium according to the invention can be preferably utilized in the method for detecting the microorganisms in the analyte. Such a method includes a step of inoculating the analyte into the second layer of the culture medium, a step of culturing the microorganisms contained in the analyte and a step of detecting the stained colony of the microorganisms according to the invention. Here, the enzyme substrate contained in the first layer of the culture medium according to the invention is a substrate to be decomposed by the enzyme of the target microorganisms. Conditions in the culturing step are not particularly limited, but 24 to 48 hours at 35±2° C. are preferred.

The culture medium according to the invention suppresses staining of the culture medium itself, and stains grown colony of the target microorganisms, and therefore the target microorganisms can be detected as the stained colony with high accuracy.

Accordingly, if the culture medium according to the invention is apprehended from another aspect, the culture medium according to the invention can be preferably utilized in a method for suppressing staining of the culture medium. Such a method includes a step of inoculating the analyte into the second layer of the culture medium, a step of culturing the microorganisms contained in the analyte and a step of detecting the stained colony of the microorganisms according to the invention. Here, the enzyme substrate contained in the first layer of the culture medium according to the invention is a substrate to be decomposed by both the enzyme of the target microorganisms and the enzyme contained in the analyte. In addition, the enzyme of the target microorganisms and the enzyme contained in the analyte may be identical or different.

Table 1 shows examples of target microorganisms, retained enzymes therein, enzyme substrates and analytes containing enzymes that can decompose the enzyme substrates. However, combinations to which the method according to the invention can be applied are not limited thereto.

TABLE 1

| Target microorganisms | Enzyme | Enzyme substrate * | Analyte |
|---|---|---|---|
| Standard bacteria | Phosphatase, Esterase | X-phosphate, X-acetate, | Raw tissues of meat, fish, |
| | | X-butyrate | vegetable or the like |
| Coliform bacilli | β-galactosidase | χ-β-galactopyranoside | Cheese, lactic acid |
| | | | bacteria beverage, |
| | | | fermented food, liver |
| Escherichia coli (E. coli) | β-glucuronidase | χ-β-glucuronic acid | Liver, oyster |
| Staphylococcus aurei | Phosphatase | X-phosphate | Raw tissues of meat, fish, |
| | | | vegetable or the like |
| Fungi (mold and yeast) | Phosphatase, Esterase | X-phosphate, | Raw tissues such as |
| | | X-acetate, X-butyrate | meat, fish and vegetable |
| Salmonella | α-galactosidase | X-α-galactopyranoside | Seeds, small intestine |
| Listeria monocytogenes, | β-glucosidase | χ-β-glucopyranoside | Liver |
| Enterococcus, | | | |
| Vibrio parahaemolyticus | | | |
| Cronobacter bacteria | α-glucosidase | χ-α-glucopyranoside | Liver |

* X represents 5-bromo-4-chloro-3-indoxyl being a blue dye group, 4-methylumbelliferryl (4MU) being a fluorescent dye group, or the like.

Specific examples of the analyte to be applied to the culture medium according to the invention include perishable food such as meat, fish and shellfish, vegetable and fruit, processed food and drink such as cheese, lactic acid bacteria beverage and fermented food, and also a clinical analyte such as feces, drinking water, fresh water, sea water and a wiping analyte in a cooking place, hospital or the like. Moreover, a culture fluid prepared by preculturing the analytes in Trypto-Soy Broth or the like, and a culture fluid prepared by further culturing the culture fluid in a culture medium for culturing microbial cells can also be used as the analyte.

### EXAMPLES

Next, the invention will be described in greater detail by way of Examples. The invention is not limited by the Examples.

### Test Example 1

(1) Preparation of Culture Medium

A total amount of a product obtained by adding each ingredient by 1 m² to 0.5 liter of purified water at a formulation shown in Table 2, and adjusting pH to 7.0 by anhydrous sodium carbonate, and warming and dissolving the resulting mixture at 95° C. for 1 minute was uniformly applied onto a 20 μm-thick polyester film having a dimension of 1 m×1 m, and the resulting material was completely dried at 65° C. The resulting product was taken as a culture medium in Comparative Example 1. With regard to culture media (Example 1 and Example 2), a total amount of a product obtained by adding each ingredient by 1 m² to 0.5 liter of purified water at a formulation shown in Table 3, and warming and dissolving the resulting mixture at 95° C. for 1 minute was uniformly further applied thereonto, and the resulting material was completely dried at 65° C. For all the culture media in Examples 1 and 2, and Comparative Example, then, a nylon meltblown nonwoven fabric (90 g/m²) was laminated on a side to which the product was applied. Further, a 100 μm-thick polyester film having a dimension of 70 mm×80 mm was adhered on the nonwoven fabric, and the resulting material was cut into 45 mm-square pieces to prepare sheet-shaped simple culture media, respectively. The detailed method for manufacturing the sheet-shaped simple culture medium was applied with reference to WO 97/24432 A.

TABLE 2
Formulation in first layer

| | (g/m²) |
|---|---|
| Peptone | 5 |
| Yeast extract | 3 |
| Bile salt | 1 |
| Dipotassium phosphate | 0.25 |
| Potassium nitrate | 0.5 |
| Sodium pyruvate | 0.5 |
| Tween 80 | 0.2 |
| X-GAL | 0.3 |
| Salmon-glucuronide | 0.3 |
| Polyvinyl alcohol * | 120 |

* Kuraray Poval 217, weight average molecular weight: 5,000 to 200,000, degree of saponification: 87 to 89%, (made by Kuraray Co., Ltd.)

TABLE 3
Formulation in second layer

(g/m²)

| | Comparative Example 1 | Example 1 | Example 2 |
|---|---|---|---|
| Polyvinyl alcohol * | (Without lamination) | 1 | 2 |

* Kuraray Poval 217, weight average molecular weight: 5,000 to 200,000, degree of saponification: 87 to 89%, (made by Kuraray Co., Ltd.)

(2) Sample Provision of Strain

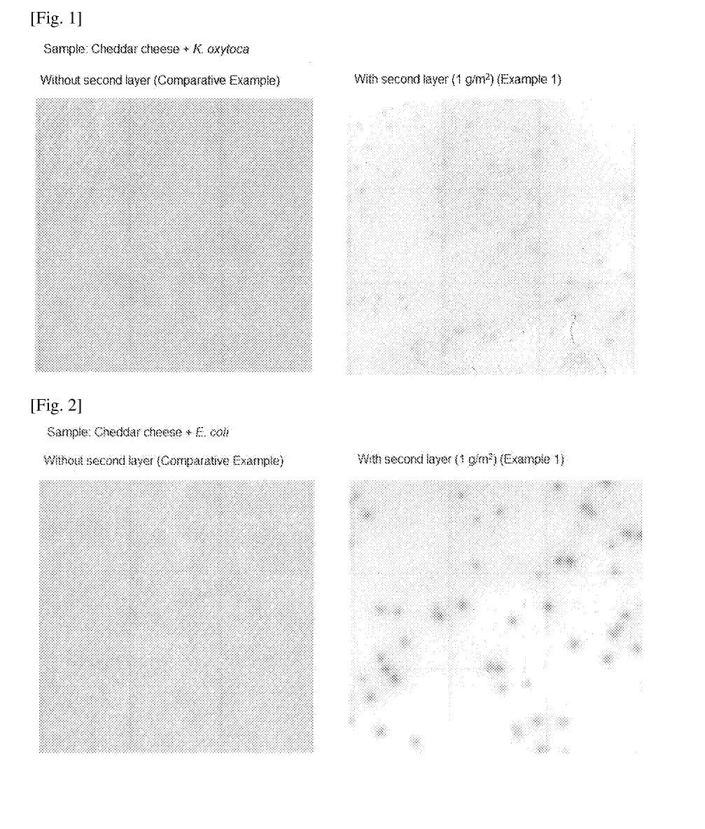

A product obtained by culturing Klebsiella oxytoca JCM 1665 and Escherichia coli NBRC 102203 each on a Trypto-Soy Agar medium for 24 hours was taken as sample bacteria. Each sample strain was suspended into sterile physiological saline using a sterile cotton swab to be a concentration corresponding to McFarland nephelometry No. 1 (about 3.0×10⁸ CFU/mL), and taken as a bacteria stock solution. Then, a solution of 10⁻⁶ CFU/mL was prepared by repeatedly diluting each bacteria stock solution in 10-fold stages by using sterile physiological saline. Separately, a milky liquid was prepared by mixing 20 g of Cheddar cheese with 90 mL of sterile physiological saline, and applying stomaching processing to the resulting mixture. In addition, an amount of the Cheddar cheese was two times an amount in the test generally conducted. Then, 1 mL of diluent of 10⁻⁶ CFU/mL of the sample bacteria was added thereto, and the resulting mixture was taken as a test sample. Then, 100 μm-thick polyester film was peeled therefrom, and the test sample was inoculated each by 1 mL into the culture media in Example 1, Example 2 and Comparative Example 1, and then the resulting material was cultured at 35° C. for 24 hours. Then, states of staining of the culture media and colony of sample bacteria were observed. The results are shown in Table 4 and FIGS. 1 to 2.

TABLE 4
Results of Test Example 1

| | | Comparative | Exam- | Exam- |
|---|---|---|---|---|
| Test sample | | Example 1 | ple 1 | ple 2 |
| Cheddar cheese + | Culture | Wholly colored | Not | Not |
| K. oxytoca | medium | | colored | colored |
| | Colony | Immeasurable | Blue | Blue |
| Cheddar cheese + | Culture | Wholly colored | Not | Not |
| E. coli | medium | | colored | colored |
| | Colony | Immeasurable | Dark blue | Dark Blue |

In the culture medium in Comparative Example 1, X-GAL was decomposed by the enzyme derived from Cheddar cheese, and the culture medium was wholly stained. Therefore, the sample bacteria that should be grown as blue or dark blue colony were unable to be distinguished at all (left in FIG. 1 and left in FIG. 2). In contrast, in Example 1, the culture medium itself was not stained, and blue colony formed by grown sample bacteria was able to be distinguished (right in FIG. 1 and right in FIG. 2). Even in Example 2 in which an amount of gelling agent in the second layer was increased to a double, the similar results were obtained.

In the present Test Example, although no direct contact of the sample bacteria (target microorganisms) was made with the first layer upon providing as the sample because the test sample was inoculated into the culture medium from the side of the second layer, the target microorganisms decomposed the enzyme substrate in the first layer to form the blue colony, and the first layer fulfilled a function as an enzyme substrate culture medium, which results were unpredictable from findings that have been so far obtained.

### Test Example 2

(1) Preparation of Culture Media

As a first layer, X-GAL Agar medium (made by Nissui Pharmaceutical Co., Ltd.) was prepared according to an attached document, and the resulting material was dispensed and solidified at a thickness of about 5 mm on a 90 mm-diameter sterile plastic petri dish. As a second layer, culture media according to the invention (Examples 3 to 8) were prepared by laminating Nutrient Agar (nutrient agar medium, made by Merck Ltd., Japan) prepared by an ordinal method or agar prepared by dissolving at 15 g/L and sterilizing the resulting product on the first layer that was previously prepared at a thickness of 1 mm, 2 mm or 5 mm. Moreover, a culture medium on which no second layer was laminated was prepared as Comparative Example 2.

(2) Sample Provision of Strain

A test sample was prepared by mixing a bacteria stock solution of Coliform bacilli and a milky liquid of Cheddar cheese in a manner similar to Test Example 1. The test sample was smeared each by 500 μL onto the culture media in Examples 3 to 8 and Comparative Example 2 from a side of the second layer. The test sample was completely absorbed into the culture media, and then the resulting material was cultured at 35° C. for 24 hours, and then states of staining of the culture media and colony of sample bacteria were observed.

The results are shown in Table 5.

TABLE 5
Results of Test Example 2

| | | | Culture medium in Comparative Example 2 | | | | | |
|---|---|---|---|---|---|---|---|---|
| | | | Culture media of present invention | | | | | |
| | | First layer | | | | | | |
| | | X-GAL Agar medium | X-GAL Agar media | | | X-GAL Agar media | | |
| | | Second layer | | | | | | |
| | | | Nutrient agar media | | | Agar | | |
| | | | Example 3 | Example 4 | Example 5 | Example 6 | Example 7 | Example 8 |
| Test sample | | None | 1 mm | 2 mm | 5 mm | 1 mm | 2 mm | 5 mm |
| Cheddar | Culture | Wholly colored | Not | Not | Not | Not | Not | Not |
| cheese + | media | | colored | colored | colored | colored | colored | colored |
| K. oxytoca | Colony | Immeasurable | Blue | Blue | Blue | Blue | Blue | Blue |
| Cheddar | Culture | Wholly colored | Not | Not | Not | Not | Not | Not |
| cheese + | media | | colored | colored | colored | colored | colored | colored |
| E. coli | Colony | Immeasurable | Blue | Blue | Blue | Blue | Blue | Blue |

In the culture medium in Comparative Example 2, X-GAL was decomposed by the enzyme derived from Cheddar cheese, and the culture medium was wholly stained. Therefore, test bacteria that should be grown as blue colony were unable to be distinguished at all. In contrast, in the culture media in Examples 3 to 8, the culture media themselves were not stained, whether or not a nutritional ingredient is contained in the second layer, and the blue colony formed by grown sample bacteria was able to be clearly distinguished. Even if the thickness of the second layer was adjusted to 5 mm, the target microorganisms decomposed the enzyme substrate in the first layer and formed the blue colony, and the first layer fulfilled a function as an enzyme substrate culture medium, which results were unpredictable from findings that have been so far obtained.

### INDUSTRIAL APPLICABILITY

The invention provides a culture medium in which staining of the culture medium itself is suppressed, even if an enzyme that can decompose an enzyme substrate to be decomposed by an enzyme of microorganisms inherently exists in an analyte. Then, the invention also provides a method for detecting target microorganisms as stained colony with high accuracy by using the culture medium according to the invention, and therefore is useful. Further, in the invention, an effect is particularly exhibited in a form of a simple culture medium being in a dry state before use, and therefore rapid and simple detection of the target microorganisms is realized, and therefore the invention is useful.

1. A culture medium for detecting microorganisms, comprising:
a first layer in which a gelling agent and an enzyme substrate are contained, and
a second layer in which a gelling agent is contained and the enzyme substrate is not contained to be laminated adjacently to the first layer,
wherein the enzyme substrate is a compound from which a dye compound can be released.

2. The culture medium according to claim 1, wherein the gelling agent comprises one kind or two or more kinds selected from the group of agar, guar gum, xanthan gum, locust bean gum, gellan gum, polyvinyl alcohol, alkylcellulose, carboxyalkylcellulose and hydroxyalkylcellulose.

3. A material for detecting microorganisms, comprising:
a culture medium according to claim 1, and
a third layer in which a porous material is contained to be laminated adjacently to the second layer on a side opposite to a side to which the first layer is adjacent.

4. A method for detecting microorganisms, comprising a step of inoculating an analyte into the second layer in the culture medium according to claim 1, a step of culturing the microorganisms contained in the analyte, and a step of detecting stained colony of the microorganisms,
wherein the enzyme substrate is a substrate to be decomposed by an enzyme of the microorganisms.

5. A method for suppressing staining of a culture medium, comprising a step of inoculating an analyte into the second layer in the culture medium according to claim 1, and a step of culturing the microorganisms contained in the analyte,
wherein the enzyme substrate is a substrate to be decomposed by an enzyme of the microorganisms and an enzyme contained in the analyte.
